(19) United States
(12) Patent Application Publication
(10) Pub. No.: US 2014/0303487 A1
(43) Pub. Date: Oct. 9, 2014

(54) Magnetic Resonance Based Method for Assessing Alzheimer's Disease and Related Pathologies

(71) Applicant: Acuitas Medical Limited, Swansea (GB)

(72) Inventors: Timothy W. James, Santa Barbara, CA (US); Kristin James, Santa Barbara, CA (US); Lance W. Farr, Swansea (GB); David R. Chase, Santa Barbara, CA (US); J. Michael Brady, Oxford (GB); James Rafferty, Godrergraig (GB); John P. Heinrich, Fort Collins, CO (US)

(73) Assignee: Acuitas Medical Limited, Swansea (GB)

(21) Appl. No.: 14/205,733

(22) Filed: Mar. 12, 2014

Related U.S. Application Data
(63) Continuation of application No. PCT/US2012/054934, filed on Sep. 12, 2012.
(60) Provisional application No. 61/534,020, filed on Sep. 13, 2011.
(60) Provisional application No. 61/596,424, filed on Feb. 8, 2012.
(60) Provisional application No. 61/639,002, filed on Apr. 26, 2012.

(51) Int. Cl. A61B 5/055; G01R 33/483; G01R 33/56

(52) U.S. Cl. CPC A61B 5/0555; A61B 5/055; G01R 33/5601; G01R 33/483
USPC 600/415; 600/410; 600/420

(57) ABSTRACT

The disclosed invention is a method for detecting indications of the presence of Alzheimer's disease (AD) and related dementia-inducing, motor-control-related pathologies, and other diseases in the human brain using a magnetic-resonance based technique for measuring fine tissue and bone textures. Specifically, the invention focuses on refinements/adaptations to a prior art magnetic resonance fine texture measurement technique that facilitates/enables pushing the detection limits closer to the cellular level, so as to be able to measure the fine scale structures and tissue changes that are known to be characteristic of the neurodegenerative processes involved in the development of these diseases.

### CROSS-REFERENCE TO RELATED APPLICATIONS

This application is a continuation of International Application No. PCT/US2012/054934 filed Sep. 12, 2012 which claims the benefit of U.S. Provisional Patent Application No. 61/534,020 filed Sep. 13, 2011, U.S. Provisional Patent Application No. 61/596,424 filed Feb. 8, 2012 and U.S. Provisional Patent Application No. 61/639,002 filed Apr. 26, 2012.

### BACKGROUND OF THE INVENTION

1. Field of the Invention

The present invention relates to the field of diagnostic assessment of changes in brain structures and tissue in response to disease progression and treatment, specifically in response to Alzheimer's Disease (AD) and related forms of dementia such as Dementia with Lewy Bodies (DLB) and Frontotemporal Dementia (FTD), as well as motor diseases such as Amyotrophic Lateral Sclerosis (ALS), and Parkinson's disease, but also in all other pathologies that involve changes to normal brain structures such as CVD, (Cerebrovascular Disease), autism, MS (multiple sclerosis), and epilepsy, as well as mental diseases and injuries associated with changes in fine brain structures.

2. Prior Art

Alzheimer's disease is a common form of dementia, a range of diseases that result in gradual loss of memory and cognitive function. The exact cause of AD is unknown and, although a variety of therapies that purport to lessen the effects of AD are available, there is at present no cure. Due to the ageing population, an AD epidemic looms, bringing with it a considerable societal and economic impact.

In general, the earlier evidence of a disease can be detected, the more options are available for its management. The definitive diagnosis for AD at present is histology, which can be performed only at autopsy. Though various in vivo diagnostics are available, there is no definitive diagnostic currently available for longitudinal use. One of the greatest unmet needs in medicine, brought on by the surge in AD cases, is an early stage, non-invasive method to detect onset/disease proclivity and monitor progression. An accurate, non-invasive technique to detect AD and other dementias would play an essential role in the development and monitoring of new therapies.

Though AD is linked to a range of chemical and morphological changes in the brain, no definitive etiology has been found. By post mortem histology, AD (and other forms of dementia, including DLB (see Richard A. Armstrong et al., “Size frequency distribution of the β-amyloid (Aβ) deposits in dementia with Lewy bodies with associated Alzheimer's disease pathology”, Neurological Sciences, December 2009, Vol. 30, No. 6, pps. 471-477)) has been associated with an increase in the load of amyloid beta plaques (clumps of fibrous proteins) in the interneuron spaces in the brain (FIG. 7) (see Rebekah Richards, “What Are Amyloid Plaques?”, www.ehow.com, undated; Catherine E. Myers, “Amyloid Plaques”, Memory Loss & the brain, 2006, www.memorylossonline.com; “Alzheimer's Disease”, www.about.com, undated; and Richard A. Armstrong et al., “Size frequency distribution of the β-amyloid (aβ) deposits in dementia with Lewy bodies with associated Alzheimer's disease pathology”, Neurological Sciences, December 2009, Vol. 30, No. 6, pps. 471-477), an increase of neurofibrillary tangles (abnormal accumulations of twisted tau protein fragments) within the neuron (see Catherine E. Myers, “Amyloid Plaques”, Memory Loss & the brain, 2006, www.memorylossonline.com; and Richard A. Armstrong, “Clustering and periodicity of neurofibrillary tangles in the upper and lower cortical laminae in Alzheimer's disease”, Folia Neuropathologica, 2008, Vol. 46, No. 1, pps. 26-31), brain atrophy and associated volumetric shrinkage in the hippocampus and cortex of the brain (see “MRI Shows Brain Atrophy Pattern That Predicts Alzheimer's”, Feb. 10, 2009, ScienceDaily, www.sciencedaily.com, 2 pps. total; An-Tao Du, Norbert Schuff, Joel H. Kramer, Howard J. Rosen, Maria Luisa Gorno-Tempini, Katherine Rankin, Bruce L. Miller, Michael Weiner, “Different Regional Patterns of Cortical Thinning in Alzheimer's Disease and Frontotemporal Dementia”, Brian. 2007 April; 130 (Pt 4): 1159-1166; “Shrinkage of Hippocampus Predicts Development of Alzheimer's”, MedPage Today, Mar. 16, 2009, http://www.medpagetoday.com/Neurology/AlzheimersDisease/13284; and Giovanni B. Frisoni, Rossana Ganzola, Elisa Canu, Udo Rüb, Francesca B. Pizzini, Franco Alessandrini, Giada Zoccatelli, Alberto Beltramello, Carlo Caltagirone and Paul M. Thompson, “Mapping Local Hippocampal Changes in Alzheimer's Disease and Normal Ageing with MRI at 3 Tesla”, Brain (2008), 131, 3266-327), myelin loss in the corpus callosum leading to axial shrinkage (see Evan Godt, “AR: MRI reveals atrophy in early AD patients”, Apr. 12, 2012, www.healthimaging.com), as well as tissue and structure degradation in other brain regions. This atrophy is most noted in the cortical gray matter and techniques have been developed that allow monitoring of the associated cortical thinning over time through use of image segmentation and registration (see An-Tao Du, Norbert Schuff, Joel H. Kramer, Howard J. Rosen, Maria Luisa Gorno-Tempini, Katherine Rankin, Bruce L. Miller, Michael Weiner, “Different Regional Patterns of Cortical Thinning in Alzheimer's Disease and Frontotemporal Dementia”, Brian. 2007 April; 130 (Pt 4): 1159-1166). Another morphological change seen in post mortem AD histology, as well as to some degree in other forms of dementia and cognitive abnormality such as autism and schizophrenia (see Steven Chance, “Cortical Hierarchy and Ageing of Cortical Minicolumns”, undated, 32 pps. total; and Steven A. Chance et al., “Auditory cortex asymmetry, altered minicolumn spacing and absence of ageing effects in schizophrenia”, Brain, 2008, Vol. 131, No. 12, pps. 3178-3192) is an upset in the normal organization of neurons and their associated axonal and dendritic fibers in the cortex of the brain. In the cortex of a healthy brain, neuronal cell bodies tend to line up in columns of some 100 cell bodies stacked roughly one above another orthogonal to the cortical surfaces. Their associated axons and dendrites form bundles running parallel to the stacked neurons (see Steven A. Chance et al., “Microanatomical Correlates of Cognitive Ability and Decline: Normal Ageing, MCI, and Alzheimer's Disease”, Cerebral Cortex, August 2011, Vol. 21, No. 8, pps. 1870-1878; Enrica Di Rosa et al., “Axon bundle spacing in the anterior congulate cortex of the human brain”, Journal of Clinical Neuroscience, 2008, Vol. 15, pps. 1389-1392; Daniel P. Buxhoeveden et al., “The minicolumn hypothesis in neuroscience”, Brain, 2002, Vol. 125, pps. 935-951; and Grazyna Rajkowska et al., “Cytoarchitectonic Definition of Prefrontal Areas in the Normal Human Cortex: I. Remapping of Areas 9 and 46 using Quantitative Criteria”, Cerebral Cortex, July/August 1995, Vol. 5., pps. 307-322). In the literature, these columnar bundles are termed “minicolumns”. The repeat distance of these grouped structures is on the order of 30-100 microns in most regions of the cortex. (The reported values in the literature are low as, by convention, the significant shrinkage inherent in fixation of brain tissue and subsequent forming of the histology slice is not corrected for, as it varies depending on the exact method used (see Enrica Di Rosa et al., “Axon bundle spacing in the anterior congulate cortex of the human brain”, Journal of Clinical Neuroscience, 2008, Vol. 15, pps. 1389-1392)). While some change in the organization (column spacing and regularity) of the columnar structure of the minicolumns is associated with normal ageing, its occurrence at an accelerated and exaggerated pace has been found through histology studies to be a sensitive indicator of AD onset and progression; column thinning occurs continuously through onset and development of mild cognitive impairment (MCI) and the columnar organization eventually virtually disappears in advanced AD and other dementias such as DLB (Dementia with Lewy Bodies) (FIGS. 3, 4) (see Steven A. Chance et al., “Microanatomical Correlates of Cognitive Ability and Decline: Normal Ageing, MCI, and Alzheimer's Disease”, Cerebral Cortex, August 2011, Vol. 21, No. 8, pps. 1870-1878; and S. V. Buldyrev et al., “Description of microcolumnar ensembles in association with cortex and their disruption in Alzheimer and Lewy body dementias”, Proceedings of the National Academy of Sciences of the Unites States of America, May 9, 2000, Vol. 97, No. 10, pps. 5039-5043) along with neuronal death. The differential temporal progression of this upset in minicolumn organization in different control regions of the cortex is thought to be an indicator of the specific pathology inducing the dementia. For instance, in AD these changes are thought to progress from the medial temporal out to the lateral temporal lobe and then forward to the prefrontal cortex.

The biophysical cascade of processes associated with AD that leads to the thinning of, and progressive disruption in, cortical minicolumns, to the deposition of amyloid beta as plaques that accumulate in inter-neuronal spaces including vasculature, and to formation of neurofibrillary tangles may also manifest as other fine structure morphological changes in brain tissue such as demyelination of axons and shrinkage of axonal tracks (see Evan Godt, “AR: MRI reveals atrophy in early AD patients”, Apr. 12, 2012, www.healthimaging.com). Another structural feature that may be disrupted as either a cause of or result of forms of dementia including AD, is the microvasculature within the cortex, which in normal brain has a characteristic spacing on the order of 30-100 microns (FIG. 6).

Besides these tissue changes, other changes within the brain tissue, may accompany AD, as it is widely believed to be part of a class of inflammatory diseases (see “Alzheimer's Disease”, www.about.com, undated) and so is expected to have a range of fine tissue changes including changes in the white matter tracks in the brain (see “Alzheimer's Disease May Originate in the Brain's White Matter”, http://www.scienceblog.com/community/older/2002/F/2002261.html).

Because the structures referred to in the discussion above—minicolumns, vasculature, inflammatory upsets, etc.—refer to very fine, repeating structures, they are often, in the parlance of biology and specifically in radiology, referred to as “texture”, as they form a cumulative pattern on a scale of multiple repeats. In the following we will use interchangeably the terms “structure” and “texture” or “structural” and “textural”, depending on what particular attribute of the structure/texture is under discussion.

Both MR (Magnetic Resonance) and PET imaging modalities are used to detect tissue changes associated with the development of AD and have been used as inclusion/monitoring criteria in clinical studies. MRI can be used to observe gross anatomical shrinkage in regions of the brain, such as the medial temporal lobe, hippocampus, and corpus callosum, which are associated with AD (see “MRI Shows Brain Atrophy Pattern That Predicts Alzheimer's”, Feb. 10, 2009, ScienceDaily, www.sciencedaily.com, 2 pps. total; and “MRI Brain Scans Accurate In Early Diagnosis Of Alzheimer's Disease”, Dec. 18, 2008, ScienceDaily, www.sciencedaily.com, 2 pps. Total). Several PET imaging agents, including PiB, florbetaben and fluorbetapir, are reported to have shown preferential uptake in regions of the brain with accumulated Aβ (see Clifford R. Jack Jr. et al., “Brain beta-amyloid measures and magnetic resonance imaging atrophy both predict time-to-progression from mild cognitive impairment to Alzheimer's disease”, Brain, 2010, Vol. 133, pps. 3336-3348; and Luiz Kobuti Ferreira et al., “Neuroimaging in Alzheimer's disease: current role in clinical practice and potential future applications”, CLINICS, 2011, Vol. 66, No. 51, pps. 19-24). In addition, PET is used together with radiotracer-tagged glucose to study effects on brain metabolism (FDG PET). However, neither of these modalities currently has the ability to see changes on the structural size level of the columnar organization of neurons in-vivo.

Currently in clinical practice diagnosis of cognitive impairment leading to AD is often made from anecdotal reports of behavior changes and by a battery of tests of cognition and memory. As such, diagnosis is often not made until the disease has resulted in significant changes to a patient's behavior. For earlier diagnosis, a reliable and specific in vivo biomarker indicating disease onset and early pathology development, or proclivity to disease advancement, is needed.

The five AD biomarkers currently in use in clinical trials evaluating therapy efficacy are (see Clifford R Jack, David S. Knopman, William J Jagust, Leslie M. Shaw, Paul S. Aisen, Michael W. Weiner, Ronals C. Petersen, John Q. Trojanowski, “Hypothetical Model of Dynamic Biomarkers of the Alzheimer's Pathological Cascade”, Lancet Neurology 2010; 9: 119-28):

1. Measurement of CSF (Cerebrospinal Fluid) concentration of tau protein, the protein found in NFTs (Neurofibrillary Tangles). Post mortem histology studies have demonstrated a link between NFT concentration and AD pathology.

2. CSF concentration of soluble Aβ₄₂ (Amyloid Beta), which varies inversely with Aβ plaque concentration in the brain and often with AD advancement.

3. PET (Positron Emission Tomography) using PiB (Pittsburgh Compound B) or other radiotracer that binds to Aβ plaques.

4. FDG (fluorodeoxyglucose) PET, which is used to image brain metabolism rate throughout the brain; metabolism is an indicator of synaptic transmission efficiency.

5. MRI measured cortical thinning/volumetric loss, especially as a longitudinal measure, correlates with brain atrophy.

Four out of five of these biomarkers present serious drawbacks to use as a routine and a longitudinal diagnostic.

Use of CSF biomarkers involves painful and invasive sample withdrawal, and therefore is not able to be used routinely longitudinally. The associated risk would preclude its use in clinical studies without clearly demonstrable patient benefit. Similar to spinal tap, this procedure involves incision through the epidural lining around the spinal column. Further, these fluid sampling biomarkers cannot differentiate signal levels by anatomic position in the brain, as is possible with imaging biomarkers. As the progress of various forms of dementia, as well as stages in pathology progression, are often distinguished by differential effects and rates of progression in different brain regions, this is a serious drawback to use of fluid biomarkers.

Along with being extremely costly, PET imaging requires use of radiotracers and positioning/calibration x-rays. This makes implementation as a routine diagnostic, and especially as a longitudinal one, problematic. Further, the role of Aβ plaques in disease etiology is not well understood; it is thought that these plaques may be incidental rather than causative in the disease process (see Mateen C. Moghbel et al., “Amyloid-β imaging with PET in Alzheimer's disease: is it feasible with current radiotracers and technologies?”, European Journal of Nuclear Medicine and Molecular Imaging, Oct. 19, 2011). (Some PET agents, including PiB, also have half-lives too short to allow practical clinical use.) Therapies that remove amyloid beta plaques from the brain have been shown to offer no improvement in, or slowing of loss of, cognition. In fact, trials of the most recent forms of such therapies have been stopped indefinitely (see “Trials for Alzheimer's Drug Halted after Poor Results”, New York Times, 6 Aug. 2012; “Alzheimer's Drug Fails Its First Big Clinical Trial”, New York Times, 23 Jul. 2012; and “Most Work Stops on Major Alzheimer's Drug”, Med Page Today, 6 Aug. 2012. http://www.medpagetoday.com/Neurology/AlzheimersDisease). Use of PET radiotracers to determine Aβ load is problematic: there is often a striking discrepancy in the measured distribution of Aβ deposits in the brain from PET radiotracer images as compared to that measured by histopathological and immunohistochemical studies, which may be due in part to the low resolution of PET imaging (2-3 mm) causing partial volume effects when used to measure structures on the order of 100 microns (see Mateen C. Moghbel et al., “Amyloid-β imaging with PET in Alzheimer's disease: is it feasible with current radiotracers and technologies?”, European Journal of Nuclear Medicine and Molecular Imaging, Oct. 19, 2011). Further, though the association of plaques with AD is well documented in the literature, plaque load does not track with cognitive decline—in some 15% of cases, elderly patients showing high amyloid beta load on autopsy have exhibited no cognitive impairment. But most tellingly, amyloid beta deposits form in the brain decades before onset of any cognitive decline, in fact, by the time symptoms occur, the load of amyloid plaque in the brain has plateaued. In some cases an amyloid burden carried well into old-age appears to have little or no effect on cognition (see “Alzheimer's memory problems originate with protein clumps floating in the brain, not amyloid plaques”, e! Science News, Apr. 27, 2010, www.esciencenews.com; and Sanjay W. Pimplikar, “Reassessing the Amyloid Cascade Hypothesis of Alzheimer's Disease”, The International Journal of Biochemistry & Cell Biology, June 2009, Vol. 41, No. 6, pps. 1261-1268). The time between plaque accumulation and onset of neurodegenerative pathology is not well known; indeed some patients with early-diagnosed high load of plaques die at old age having shown no signs of dementia. Further, therapies targeting the amyloid beta protein deposits have to date demonstrated no efficacy in slowing or reversing cognitive impairment (see “Trials for Alzheimer's Drug Halted after Poor Results”, New York Times, 6 Aug. 2012; and “Alzheimer's Drug Fails Its First Big Clinical Trial”, New York Times, 23 Jul. 2012).

The fifth diagnostic modality mentioned above, (MR) Magnetic Resonance, avoids the problems of invasiveness of these other diagnostic modalities. It can be used longitudinally and its cost is approximately one quarter that of PET imaging. Cortical thinning in response to brain atrophy, which can be measured using MR imaging, has been noted in recent research to be a relatively sensitive indicator of AD progression (see Clifford R Jack, David S. Knopman, William J Jagust, Leslie M. Shaw, Paul S. Aisen, Michael W. Weiner, Ronals C. Petersen, John Q. Trojanowski, “Hypothetical Model of Dynamic Biomarkers of the Alzheimer's Pathological Cascade”, Lancet Neurology 2010; 9: 119-28). Although both CSF tau and MR imaging are predictive of future conversion from MCI to AD, the predictive power of structural MRI is found to be greater.

As volumetric shrinkage is clearly associated with brain atrophy, probing the cortex and hippocampus to tag earlier changes in the neuronal organization underlying the volumetric shrinkage/cortical thinning, before these changes become magnified, offers the hope of earlier and more sensitive measure of disease. In addition, the capability for monitoring on a fine scale, other inflammatory tissue changes, plaque deposits, myelin degradation and fine-scale morphology changes brought about by brain tissue atrophy and associated shrinkage, provides a sensitive marker for the brain changes attendant with MCI and AD.

Another disease that can cause dementia is CVD (Cerebrovascular Disease), which induces cognitive impairment as a result of reduced blood flow through blocked vessels leading to brain tissue. It is difficult in many cases to differentiate dementia caused by a disease such as AD from that caused by CVD. As the appropriate therapies differ, being able to identify the underlying disease would be extremely useful in managing patient care.

Another difficulty in assessing brain function in normal as well as diseased brains arises due to a lack of ability to, in vivo, determine the boundaries of the various control regions of the cerebral cortex or the different Brodmann's areas of which these are comprised. Such ability would greatly aid data interpretation in brain function studies, such as those performed using, for example, FMRI (Functional Magnetic Resonance Imaging).

U.S. Pat. No. 7,932,720 enables measurement of biologic textures too fine to be resolved by conventional MR imaging, providing a quantitative measure of the characteristic spatial wavelengths of these textures. In its simplest form the method consists of acquiring finely-sampled spatially-encoded MR echoes along an axis of a selectively-excited inner volume positioned within the tissue region of interest and signal analysis to yield a spectrum of textural wavelengths within various regions along the axis of the selected tissue volume.

After analysis, the data can be plotted in various forms to allow comparison of spectra from any ROI (Region of Interest) along the sampled volume, as well as for comparison of spectra from different subjects. One method for plotting data is to assign color to specific wavelength ranges and map the variation in predominant structural wavelengths in spectra at continuous intervals along the length of the prism. This mapping technique is described in U.S. Pat. No. 7,309,251 entitled “Representation of Spatial-Frequency Data as a Map”. Using this technique one can map any quantity derived from the structural spectra at successive regions along the prism length. Two other possible methods of plotting the data are shown in FIGS. 8 and 9. FIG. 8 shows a method of plotting spectra by overlap of spectra obtained by centering an analysis window at successive, or closely-spaced, points along the long axis of the prism. This same data can also be plotted as a spectrogram; in the example shown in FIG. 9, the horizontal axis is distance along the prism and the vertical axis is wavelength. A color-coding scale, in this example shown in the bar to the left of the plot, is used to set the scale for the confidence levels at each wavelength.

Additional prior art refinements to the magnetic resonance fine texture measurement technique include: averaging in the complex domain to provide significant noise reduction as compared to averaging of signal magnitude; recording multiple individual echoes allows determination of the statistical significance of the various peaks present in the resulting structural wavelength spectra (FIG. 10) with subsequent averaging in the complex domain providing significant noise reduction as compared to averaging of signal magnitude (see David R. Chase et al., “fineSA Statistics and Repeatability Analysis”, Apr. 6, 2011, 16 pps. total).

### BRIEF DESCRIPTION OF THE DRAWINGS

FIG. 1 is an example of data from a selected region in the corpus callosum and display of information from 3 separate regions of interest along the prism in the form of frequency spectra in accordance with embodiments of the present invention.

FIG. 2 is an example of data taken along the AP direction at the top of the brain stem to show the ability to obtain high-resolution data even in regions of the brain associated with high cardiac-induced motion.

FIG. 3 is an image, from histology showing the variation in columnar organisation of neurons in the human brain with age. The changes seen with normal ageing are accelerated in the case of Alzheimer's disease and other forms of dementia and brain pathology, as reported in the literature. Image from Buxhoeveden D P, Casanova M F Brain 2002; 125:935-951.

FIG. 4 is a histology image stained to show A) the organization of the neurons in columns within the cerebral cortex and another B) stained to show the organization of the myelinated bundles of the axons associated with the cell body columns. The organization of these two components of the neuronal structure is seen from the photographs. From Rajkowska et al. Cereb. Cortex. 1995, 307-22.

FIG. 5, on the left, is an image from Steven A. Chance et al., “Microanatomical Correlates of Cognitive Ability and Decline: Normal Ageing, MCI, and Alzheimer's Disease” (Cerebral Cortex, August 2011, Vol. 21, No. 8, pps. 1870-1878) of histology illustrating the upset in minicolumn organization in normal, MCI, and AD cases, respectively, and the corresponding structural spectra obtained by applying the data analysis portion of the prior art magnetic resonance fine texture measurement technique to these images as an illustration of the changing spectra indicating progression of the disease through neuronal minicolumn disruption. It shows the organization of neuron bundles into “minicolumns”, in a normal brain (A, B) slight disruption in the columnar structure with onset of MCI (C, D) and major disruption in the columnar structure with onset of AD (E, F). The spectra on the right correspond with each histology image, showing a structural spectrum obtained from each image using the fineSA analysis to yield a structural frequency spectrum.

FIG. 6 is a histology image from slice taken through fold of the neocortex stained to show vasculature. The thin black lines are the capillary network and the thicker ones the feeder vessels. (From Steven Chance, Nuffield Department of Clinical Neurosciences, University of Oxford.)

FIG. 7 is a histology section showing amyloid plaque deposition in the parahippocampal gyrus of an Alzheimer's patient.

FIG. 8 is an example of a set of spectra from brain tissue generated from data taken at 2 mm intervals along the length of the prism and plotted overlaid on one chart with color-coding to show the position of a spectrum along the prism. The dotted lines, starting from the figure bottom, are the mean noise level, +68.3%, +95.4%, and +99.7% confidence intervals obtained from statistical analysis of the repeat MR echoes (see David R. Chase et al., “fineSA Statistics and Repeatability Analysis”, Apr. 6, 2011, 16 pps. total).

FIG. 9 is an example of a spectrogram showing spectrum vs. position along a selected region of interest of the selectively excited internal volume axis. The horizontal axis is position and the vertical axis is wavelength. In one possible embodiment, color would be used to represent spectrum intensity at different wavelengths with, for example, cool colors for the longer wavelength end of the spectrum and warmer colors at the shorter wavelength end.

FIG. 10 shows in the top image an intensity profile and in the bottom a spectrum derived from linear combination of MR echoes. Statistics obtained from the 200 repeat echoes were used to plot the mean noise, the +68.3%, the 95.4%, and the 99.7% confidence intervals seen in the bottom image.

FIG. 11 is a schematic illustration showing prism positioning such that the long axis of the prism is oriented along a curved section of tissue (such as cortex) containing repeating structures (such as neuron columns and fiber bundles) and further is oriented such that it intersects this structure at angles either side of orthogonal. In this way, a differential measurement can be made by comparing the textural/structural spectra obtained from successive ROI's selected along the prism length, thus providing a check on the structural separation measured from the spectra and also providing increased sensitivity to changes in the structural separation.

FIG. 12 shows MR reference images showing the positioning of a prismatic volume within the tissue of interest as used for acquiring structural wavelength data. The example given here shows the prism positioned along the top of a fold in the prefrontal cortex.

FIG. 13 shows acquisition of MR signal using a range of gradient angles along the prism length in order to ensure optimal alignment of the acquisition axis relative to the columnar organization of neuronal and axon bundles or other tissue structure for some portion of the acquisition. The specific angles would follow a spiral path or other similarly defined range of values.

FIG. 14 is a schematic of an example MR pulse sequence for acquiring T1 contrast data in the cortical regions or in other small regions of the brain to gain contrast between high fat content materials such as the myelin coating around axons and surrounding high water content substances. PSSE (Partial Symmetric Spin Echo) refers to the fact that the data is acquired using partial Fourier acquisition of a symmetric spin echo. This allows earlier spin echo time and thus earlier trailing edge data acquisition of the high frequency structural data of interest.

FIG. 15 is a schematic of example MR pulse sequence for acquisition of T2* contrast data in the cortex and surrounding brain regions. By acquiring data later in time to avoid FID leakage, contrast develops between blood in the vasculature and the surrounding tissue because the iron in blood causes rapid decay of its MR signal; it appears dark against a lighter background from the surrounding tissue. Putting the spin echo before k0 allows greater development of T2* contrast by the time the high-frequency k values of interest are recorded, before T2 decay has significantly reduced the signal. This is the PEASE (Partial Early Asymmetric Spin Echo) sequence.

FIG. 16 is an image showing subject on scanner bed with head positioned within the stabilizing cradle.

FIG. 17 is a schematic of example MR pulse sequence for acquisition of T2 contrast data such as would be used to see inflammatory tissue response. By positioning the spin echo near the high k values of interest, but late enough in time for T2 contrast to develop, signal to noise is maximized. This is the PASE (Partial Asymmetric Spin Echo) sequence.

### DETAILED DESCRIPTION OF THE PREFERRED EMBODIMENTS

The current invention consists of adaptations/refinements to U.S. Pat. No. 7,932,720, to facilitate application of this prior art to brain pathology, specifically to the etiology attendant with onset and development of AD and other associated dementias, though the refinements also can be applied to probing regions of the brain to measure many other pathology and trauma-induced tissue effects.

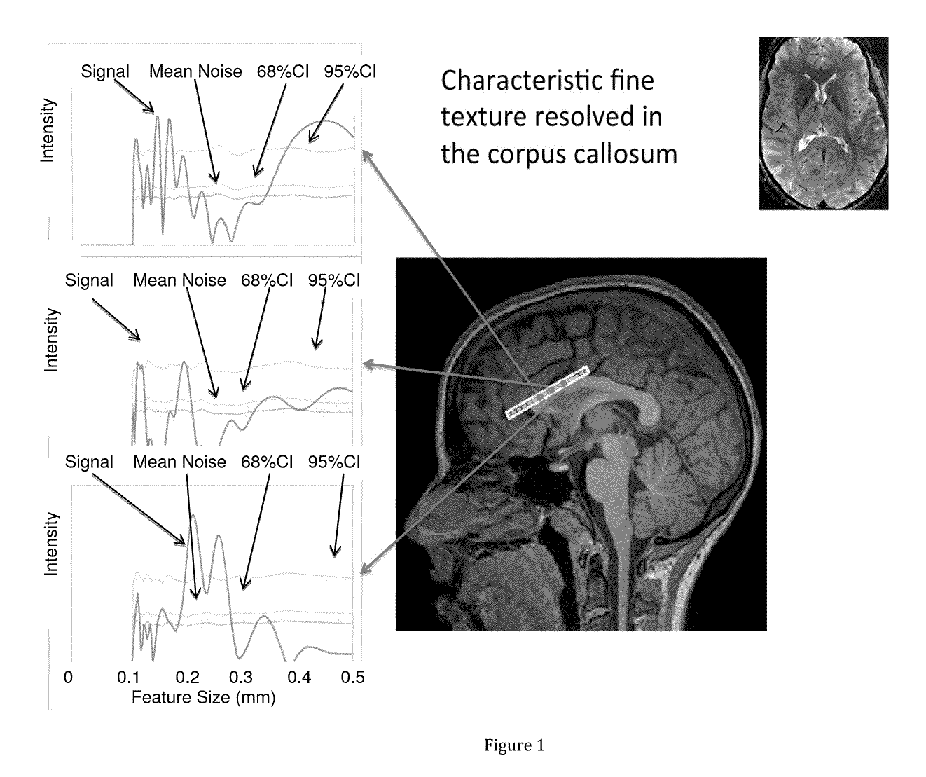

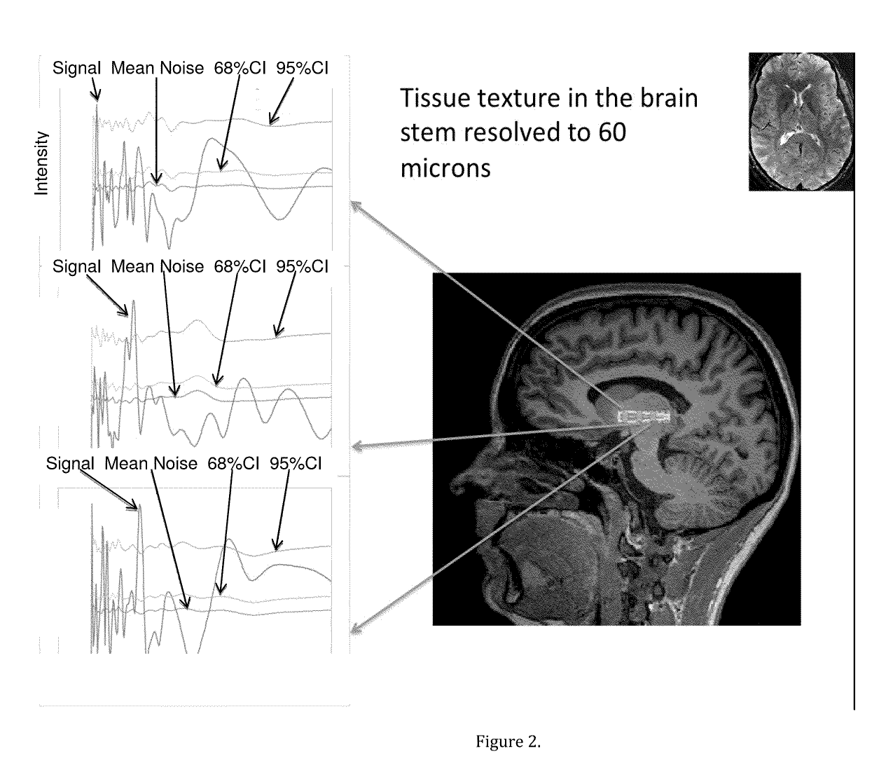

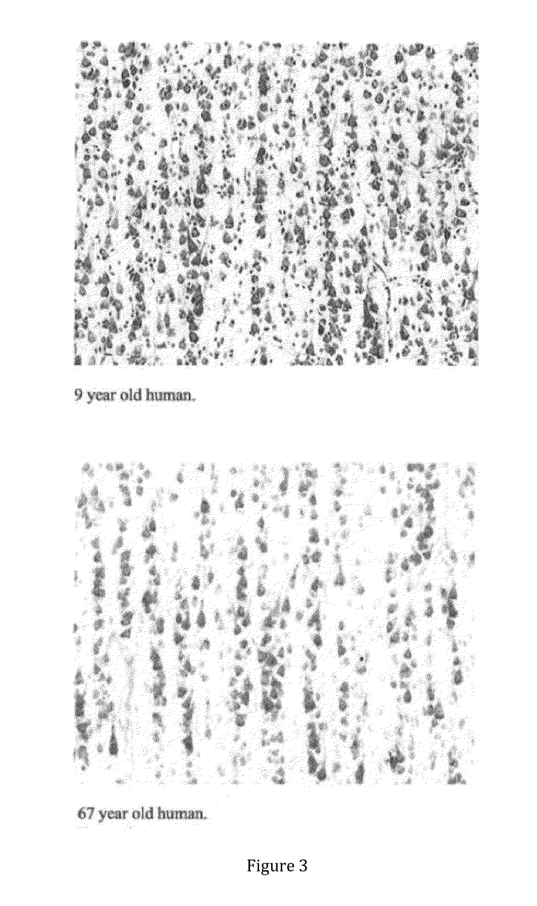

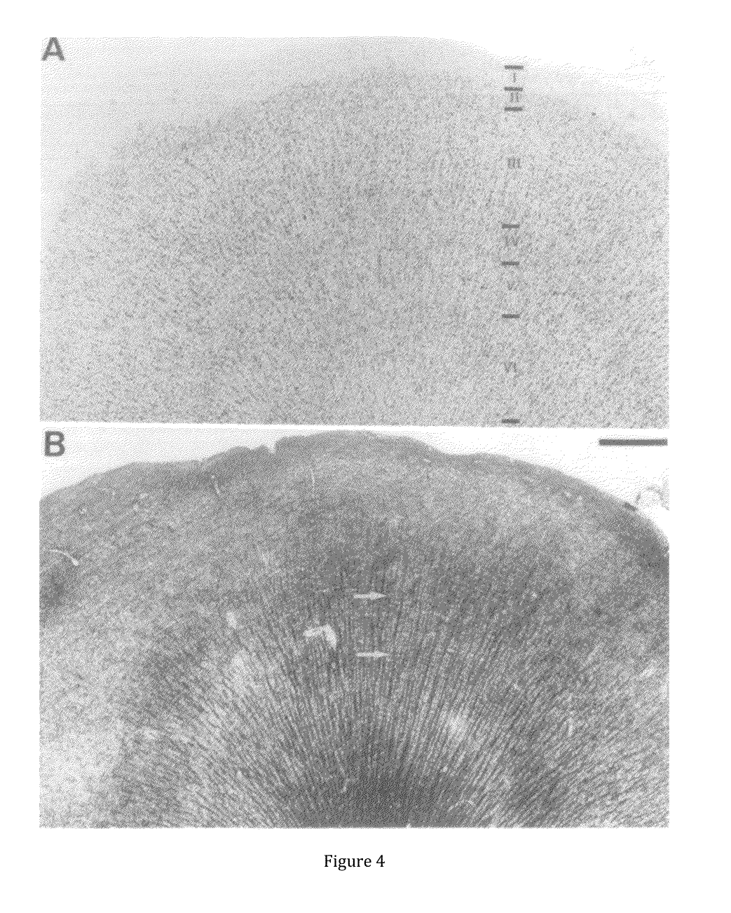

FIGS. 1 and 2 show the magnetic resonance fine texture measurement technique applied to brain. Structural wavelength spectra are generated from the indicated regions of interest along an axis of the selectively excited inner volume.

In order to define terminology for what follows, and with reference to the prior art magnetic resonance fine texture measurement technique, an internal volume in the anatomy of interest is excited by proper sequencing of magnetic field gradients and RF (Radio Frequency) pulses. Acquisition of the finely sampled 1D data is enabled by application of a readout gradient along a selected direction within the volume.

The inner volume can be defined in a multitude of shapes and sizes; as one example, by application of orthogonal magnetic gradients and subsequent application of two RF pulses of properly selected bandwidth, a rectangular prism-shaped volume can be excited. By application of a readout gradient, for example along the long axis of the prism, finely sampled echo data can be acquired along this axis. Although a rectangular prism is one possible volume with which to acquire data, many other volumes are possible.

The readout gradient defines the direction of echo data acquisition. The moniker “readout gradient direction” may be used interchangeably with “acquisition axis” or “direction of data acquisition” or “data acquisition direction” or “acquisition direction” in the following. Additionally, to specify the volume of tissue within which the MR data is excited “selectively-excited inner volume”, “inner-volume”, and “acquisition volume” are also used interchangeably in the following.

Because the prior art magnetic resonance fine texture measurement technique is applicable to texture change of a size nearing cellular dimensions, with suitable adaptation it can be used to see the earlier changes to neurons that presage brain atrophy in dementia and other pathologies, by measurement of changes in the cortical minicolumn organization and other attendant tissue changes.

By appropriate adaptation, the prior art magnetic resonance fine texture measurement technique can be used to provide quantitative information of finer tissue changes than would be visible with MR imaging, over a large range of pathologies. For example, the change in organization of the cortical minicolumns, which appears to be a sensitive indicator of cognitive impairment in AD and other dementias and brain pathologies, can be measured and quantified and the eventual degradation and randomization of these structures seen clearly as loss of structural coherence; tissue changes, such as those underlying the atrophy associated with hippocampal shrinkage or the degradation of the white matter tracts in the corpus callosum attendant with disease can also be assessed and monitored using these same refinements; another application is to assess changes in microvasculature in response to a range of diseases; inflammatory effects, which are attendant with a range of brain pathologies, can be assessed as they induce textural change in tissue and in the structural organization of vasculature; another application would be to assess the degradation in white matter seen in MS (Multiple Sclerosis) and other degenerative brain diseases; another application is to assess the spacing of plaques and tissue changes associated with their deposition in intercellular tissue in various regions of the brain and within blood vessels. Focusing the acquisition prism on regions very small in extent, such as the cortex, hippocampus, the white matter tracts adjacent to the cortex, the corpus callosum, or the parahippocampal gyrus, for example, combined with use of appropriate contrast, can reveal tissue changes attendant with a range of additional pathologies. Cerebrovascular disease, leads to dementia through blocking of blood flow through the cerebral vascular system. Assessment of vasculature organization and integrity can differentiate whether cognitive impairment is due to CVD (Cerebrovascular Disease) in dementia as opposed to AD or other diseases, or some combination of multiple etiologies. For monitoring CVD, the invention can be used in combination with sequences designed to provide contrast in with vasculature, such as T2* contrast sequences, or with sequences designed to provide contrast with vasculature and an indication of blood flow such as BOLD (Blood Oxygenation Level Dependent) MR sequences.

There is a strong correlation observed between cognition deficits and cortical thinning/volumetric measures as measured using MR imaging (see Clifford R Jack, David S. Knopman, William J Jagust, Leslie M. Shaw, Paul S. Aisen, Michael W. Weiner, Ronals C. Petersen, John Q. Trojanowski, “Hypothetical Model of Dynamic Biomarkers of the Alzheimer's Pathological Cascade”, Lancet Neurology 2010; 9: 119-28; An-Tao Du, Norbert Schuff, Joel H. Kramer, Howard J. Rosen, Maria Luisa Gorno-Tempini, Katherine Rankin, Bruce L. Miller, Michael Weiner, “Different Regional Patterns of Cortical Thinning in Alzheimer's Disease and Frontotemporal Dementia”, Brian. 2007 April; 130 (Pt 4): 1159-1166; “Shrinkage of Hippocampus Predicts Development of Alzheimer's”, MedPage Today, Mar. 16, 2009, http://www.medpagetoday.com/Neurology/AlzheimersDisease/13284; and Giovanni B. Frisoni, Rossana Ganzola, Elisa Canu, Udo Rüb, Francesca B. Pizzini, Franco Alessandrini, Giada Zoccatelli, Alberto Beltramello, Carlo Caltagirone and Paul M. Thompson, “Mapping Local Hippocampal Changes in Alzheimer's Disease and Normal Ageing with MRI at 3 Tesla”, Brain (2008), 131, 3266-327). By positioning the selectively excited inner volume within various regions of interest within the cortex, this invention can be used in conjunction with these atrophy measurements to provide information on the finer-scale changes underlying brain shrinkage, offering the possibility of earlier diagnosis, as well as development of correlation between microscopic and macroscopic changes. In addition, positioning the cortex within the white matter also can provide corresponding information on white matter changes attendant with the brain atrophy and pathology development. By providing higher resolution structural information than is available with standard MR imaging both sensitivity to earlier disease stage and disease specificity can be increased.

The technique can be applied on its own, or can be part of a workup or existing scan for pathology-induced changes such as atrophy, lesions, or vasculature changes, adding to the resultant diagnostic information.

Because many of the textural changes attendant with brain pathology entail changes at very small dimensions, sensitive measurement of tissue changes within small, well-defined regions of the brain, such as the cortex, the hippocampus, the corpus callosum, combined with high contrast, would help provide a more complete understanding of the etiology at play in the various forms of dementia and other pathologies or trauma-induced injuries and can be used as a diagnostic and assessment tool for disease and for determining the effect of therapy.

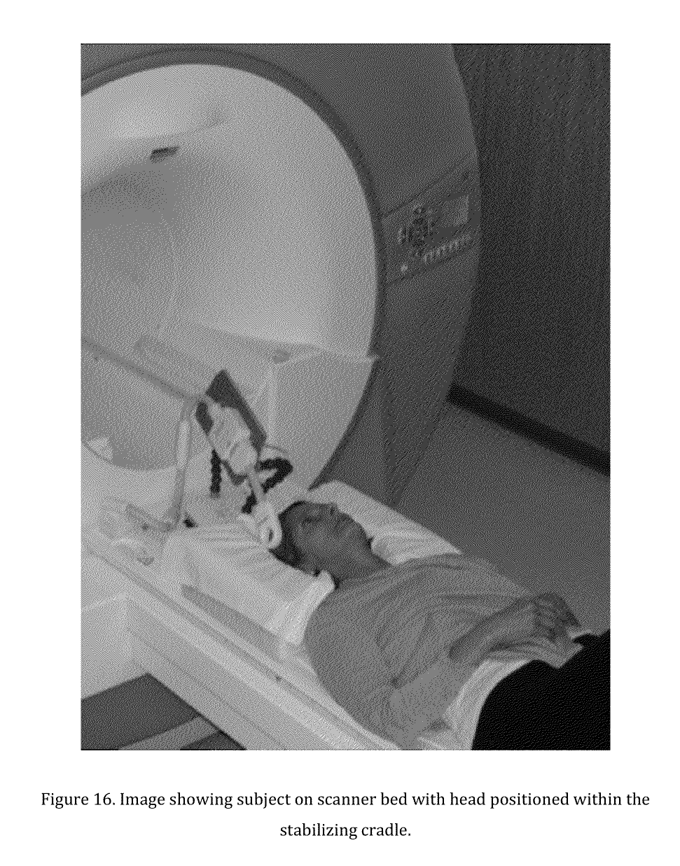

To monitor changes in narrow or small regions, proper definition of the cross-sectional size and shape of the selectively excited inner volume, as well as patient stabilization, is required. It is necessary to ensure that the selectively excited inner volume is positioned along a useable ROI completely within the targeted tissue and remains positioned within this tissue for the duration of data acquisition. As an example, the range of cortical thickness in the human brain is on the order of approximately 2-4 mm, the cortical minicolumns extending through a portion of this thickness. Therefore, to focus measurement on this tissue, a rectangular cross section on the order of 1 mm in height and 2 mm thickness can be used to fit within the cortex, yet not be so small as to seriously compromise signal amplitude. To maintain the positioning of the selectively excited internal volume within the tissue region of interest, for example in the cortex, patient motion must be held to a minimum. For this purpose, we have developed a system for subject stabilization, consisting of a head cradle that holds the patient firmly, yet comfortably in position. In its current embodiment this fixture consists of an anatomically shaped cradle made of fiberglass and cushions that surround the head and, once the subject is positioned, expand to conform to the subjects head for comfortable stabilization (FIG. 16).

In addition to this head stabilization fixture, a procedure for data acquisition has been developed by which high-resolution 3D positioning reference images are acquired both before and after each data acquisition sequence to ensure the prism has remained within the tissue of interest. If there is any displacement, the prism is repositioned within the tissue region of interest prior to the restart of data acquisition.

By tailoring the cross sectional dimensions of a rectangular cross-section volume to fit within the cortex, with some allotment to allow for small patient motion, sampling of cortical tissue can be achieved.

Positioning the internal volume to run along the top of a cortical fold maximizes the ROI along which the prism cross section stays within the cortex and maximizes the chance that the cortical minicolumn structure will be aligned close to perpendicular to the acquisition direction so as to maximize structural signal.

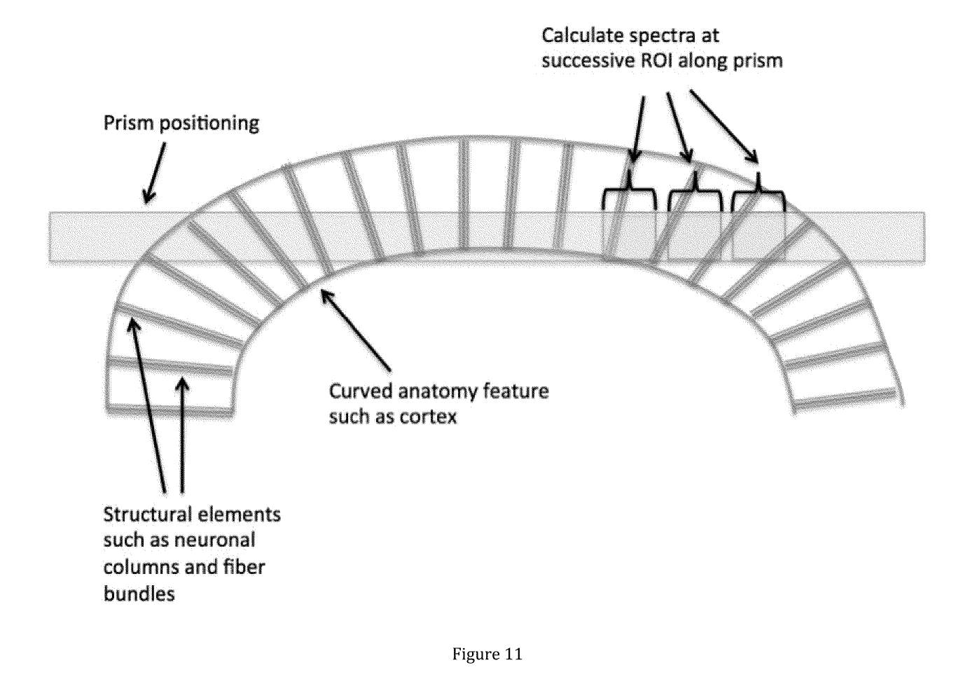

The volume can further be positioned such that its long axis is oriented along a curved section of tissue (such as cortex) containing repeating structures (such as cortical minicolumns) and further is oriented such that it intersects these structures both orthogonally and at angles either side of orthogonal. In this way, a differential measurement can be made by comparing the structural spectra obtained from multiple ROI's selected at different points along the acquisition direction. Reference images allow correlation of each spectrum obtained with the position in the brain tissue of the ROI from which the spectrum data was derived; the variation in structural wavelength spectra at each ROI should be due in large part to the variation in the angle of the structures relative to the acquisition direction. This variation can be used to provide information on the separation of structures and their overall organization. (FIG. 11). Subsequent mathematical analysis yields improved information on the state of the organization of the targeted structures.

As in other tissue applications, a major benefit of the invention is that, because it is specifically a novel way of gathering and analyzing MR data, it can be run on top of a large range of current MR imaging and contrast-generation techniques, both endogenous and exogenous. Native T1, T2, T2* contrast, BOLD (Blood Oxygenation Level Dependent) imaging which highlights vasculature, to highlight CVD (Cerebrovascular Disease) pathology and Aβ deposition in blood vessels, DTI (Diffusion Tensor Imaging), ASL (Arterial Spin Labeling), Gadolinium and other introduced contrast agents, and cardiac phase spectroscopy. Application of the technique is limited only by the physics of the signal generation.

As with other tissue types, measurement in brain tissue can be made by selecting MR parameters to generate contrast between different structures or tissues thus highlighting signal differences arising from, for example, high water content tissue such as vasculature against high fat content tissue, such as the myelin sheaths surrounding the bundled axons in the ways described below.

However, a problem arises in defining very small regions for MR excitation, such as the small cross-section volumes required to fit within the cortex or other small regions of the brain. For example, selective excitation of rectangular prism inner volumes for data acquisition is accomplished in MR scanners by applying two intersecting slice-selective RF pulse excitations in the presence of magnetic field gradients. When the inner volumes are of sufficiently small cross section, or the slices selected by the gradients are sufficiently thin, the profile of the 180° pulse slice select deviates significantly from an ideal rectangular intensity profile, leading to a non-trivial portion of the intended 180° slice selection volume being excited by other than a simple refocusing 180° pulse. The material in this off-180° condition will then have a non-trivial transverse magnetization and will produce free induction decay signal which is then encoded in the readout gradient. Signal from outside the desired volume, which is not pre-encoded, produces a large signal at the beginning of the echo, on the start of the leading edge of the echo readout, corrupting the signal from the intended inner volume. (The echo is read out in time.) To avoid contamination from this signal, which decays rapidly in time across the echo, data can be taken from trailing edge of the echo. For purposes of this document (and in general parlance) the center of the echo will be defined to fall at k0, with the leading edge occurring earlier in time and the trailing edge later.

However, because the trailing edge of the echo is later in time, signal amplitudes are lower and T2 and T2* effects induce more signal decay. In order to acquire prism data with T1, T2, and T2* contrast while still maintaining high signal to noise, we have designed pulse sequences that maximize the required contrast and signal while reducing or eliminating the effects of the improperly-encoded 180° pulse by recording only trailing edge data.

Specifically, we are using T1 and T2* contrast to assess the structure of cortical minicolumns by providing contrast to highlight the myelination surrounding the axons in the column (see David R. Chase et al., “fineSA Statistics and Repeatability Analysis”, Apr. 6, 2011, 16 pps. total) and to highlight micro-vasculature in the cerebral cortex and surrounding white matter, respectively.

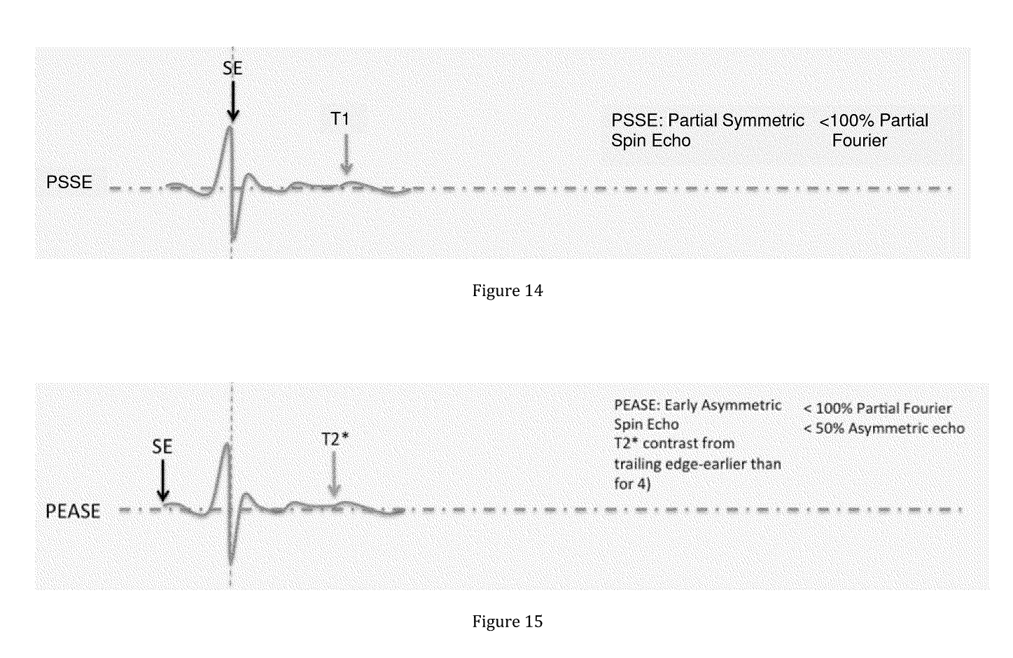

The sequence developed to highlight myelin is a PSSE acquisition pulse sequence (Partial Symmetric Spin Echo) for which partial Fourier acquisition of a symmetric spin echo is used to allow a shorter echo time and hence a stronger signal at the k values of interest. This allows acquiring of T1 contrast data in the cortical regions or in other small regions of the brain to gain contrast between high fat content materials such as the myelin coating around axons and surrounding high water content substances. PSSE (Partial Symmetric Spin Echo) refers to the fact that the data is acquired using partial Fourier acquisition of a symmetric spin echo (FIG. 14).

The sequence developed to highlight vasculature using T2* contrast is a PEASE (Partial Early Asymmetric Spin Echo) acquisition pulse sequence for which the k values of interest fall at a late time relative to the spin echo. By acquiring data from the trailing edge of the echo to avoid FID leakage, contrast develops between blood in the vasculature and the surrounding tissue because the iron in blood causes rapid decay of its MR signal; it appears dark against a lighter background from the surrounding tissue. Putting the spin echo before k0 allows greater development of T2* contrast by the time the high-frequency k values of interest are recorded, before T2 decay has significantly reduced the signal. This is the PEASE (Partial Early Asymmetric Spin Echo) sequence (FIG. 15).

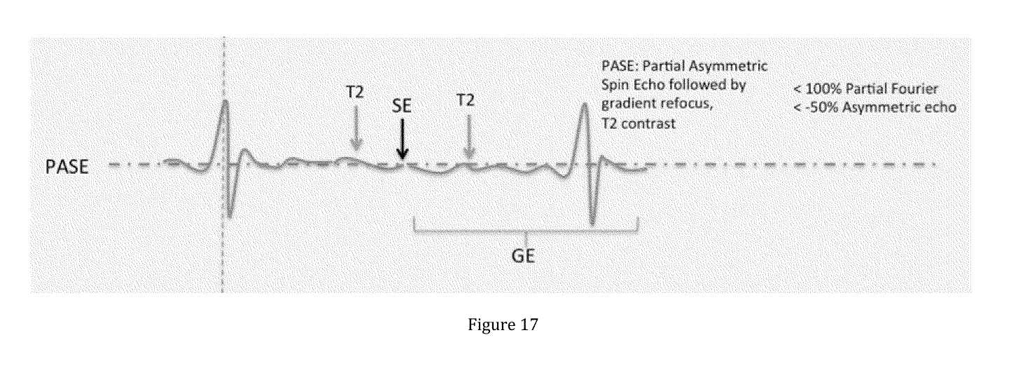

A third sequence has been developed to highlight structure linked to development of inflammatory processes, which are often imaged with T2 contrast. By positioning the spin echo close to the k-values of interest, but late enough in time for T2 contrast to develop, signal to noise is maximized. This is the PASE (Partial Asymmetric Spin Echo) pulse sequence shown in FIG. 17.

In addition to these sequences, a surface coil has been used for signal acquisition when the cortical region under study is near the skull, and placed directly against the head of the subject so as to maximize signal to noise. Selection of a cortical region in close proximity to this coil results in a higher signal than would be available from a standard multi-element (non-surface) coil assembly.

In addition, the technique is applicable independent of MR scanner type or field strength and as such can be run on both clinical and preclinical scanners.

In application to dementia prediction, diagnosis, and monitoring, the technique can be used to obtain differential signals both spatially from the different brain regions, as well as temporally by longitudinal measure, to monitor the spatial and temporal progression of the pathology and hence obtain information on whether the disease at play is AD or some other form of dementia-inducing pathology.

For example, in AD volumetric changes attendant with atrophy appear first in the medial temporal lobe and then progress to the lateral temporal, and finally in advanced AD, the frontal lobe. The rate of degeneration has been linked also to cognition impairment (see Clifford R Jack, David S. Knopman, William J Jagust, Leslie M. Shaw, Paul S. Aisen, Michael W. Weiner, Ronals C. Petersen, John Q. Trojanowski, “Hypothetical Model of Dynamic Biomarkers of the Alzheimer's Pathological Cascade”, Lancet Neurology 2010; 9: 119-28). Hence monitoring the regions in the brain known to be affected in AD pathology, and following the advancement of columnar degradation and other tissue changes to monitor spatial progression and rate of change can yield significant information pertinent to disease progression and therapy staging. Differential measures from other cortical regions, say, affected at differing rates by disease progression can be compared for verification of diagnosis or monitoring of response to treatment.

Another target for spatial and temporal differentiation in spectral signatures in the brain is correlation with observed alterations in gray and white matter tissue signal intensity attendant with aging and cognitive impairment (see D H Salat, S Y Lee, A J van der Kouwe, D N Greve, B Fischl, H D Rosas, “Age-Associated Alterations in Cortical Gray and White Matter Signal Intensity and Gray to White Matter Contrast”, Neuroimage. (2009), 48(1): 21-28). Adaptations of the prior art magnetic resonance fine texture measurement technique to brain would allow assessment and tracking of structural changes underlying this intensity variation.

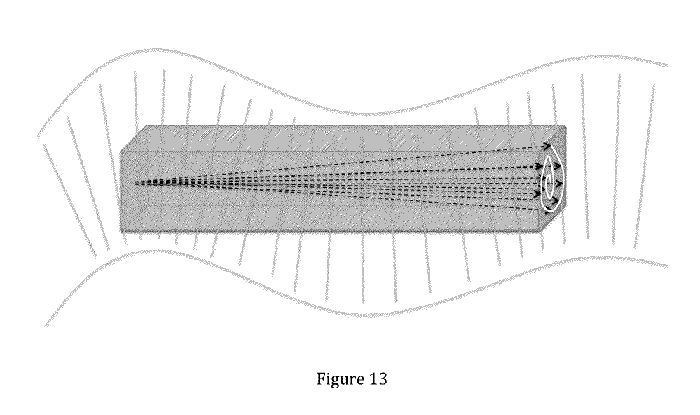

In addition, refinements to the data acquisition sequence can be used to advantage depending on the tissue targeted. For example, for assessment of cortical minicolumns or other relatively ordered, non-isotropic textures, there is a preferred alignment, relative to the orientation of the fine texture of interest, of the readout gradient angle—the direction along which echoes are acquired. As this preferred alignment angle might vary at different locations along the structure under study, an adaptation of the prior art magnetic resonance fine texture measurement technique to this situation would be to acquire successive echoes at a range of gradient angles relative to the tissue. In this way preferred alignment with the fine structures under study would occur within some band of the range of gradient angles used for data acquisition. The angular range used could, for example, over successive echo acquisitions, map out a spiraled trajectory (FIG. 13), though other criteria to determine the range of successive gradient angles can be used.

One possible means of increasing the sensitivity to cortical minicolumn organization is to position the sampling volume used for data acquisition (a prism or other selectively excited internal volume) such that the direction along which data is taken traverses a curved section of the cortex so that it intersects the columnar structure at a continually varying angle along its length. The change in wavelength spectrum with position should reflect this angular variation in a way mathematically related to the spacing of the minicolumns and their overall organization. Sampling of this variation in wavelength spectrum along the sampled length thus provides a means to determine the spacing and obtain information on the degree of order of the minicolumns—i.e. an additional measure of pathology advancement. Optimum positioning for minicolumn organization assessment would be such that the acquisition direction intersects repeating structures such as minicolumns at 90° and at angles on either side of 90°. This method is depicted in FIG. 11.

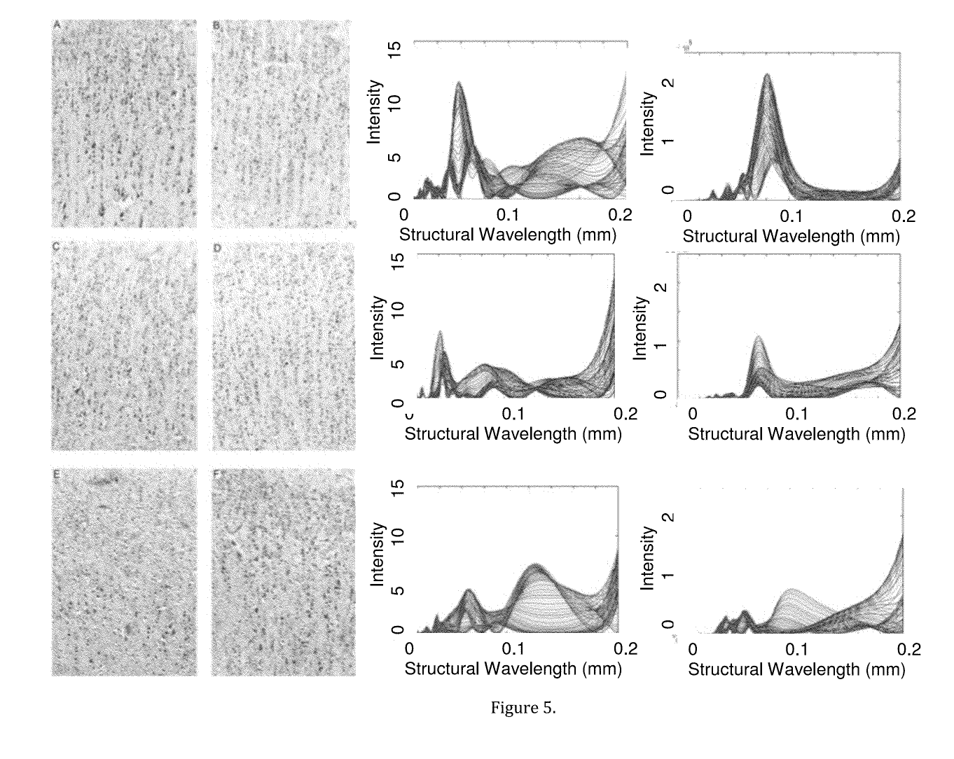

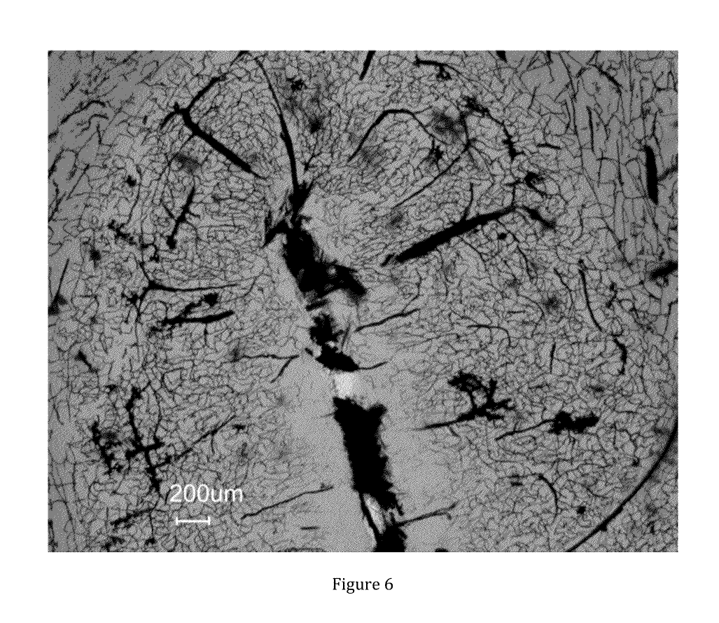

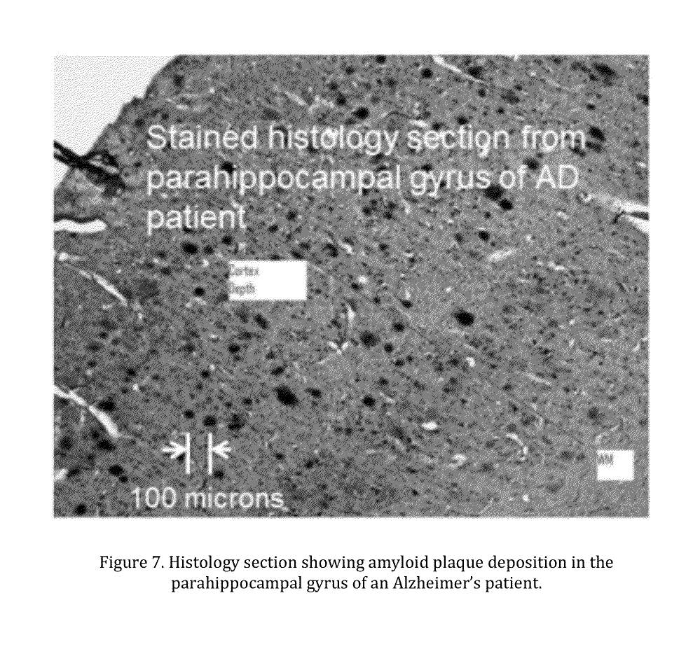

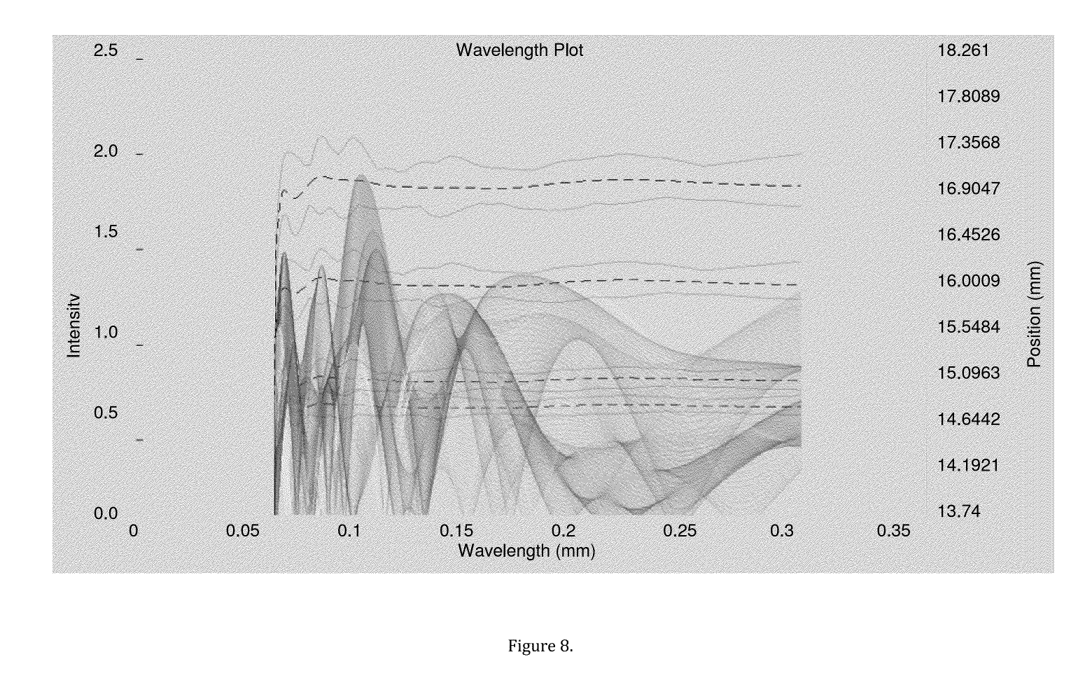

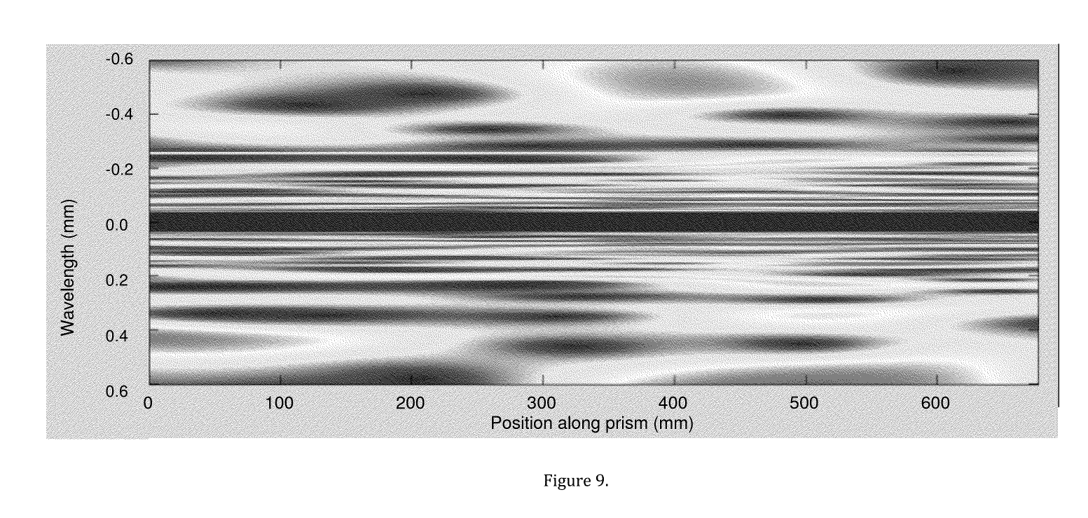

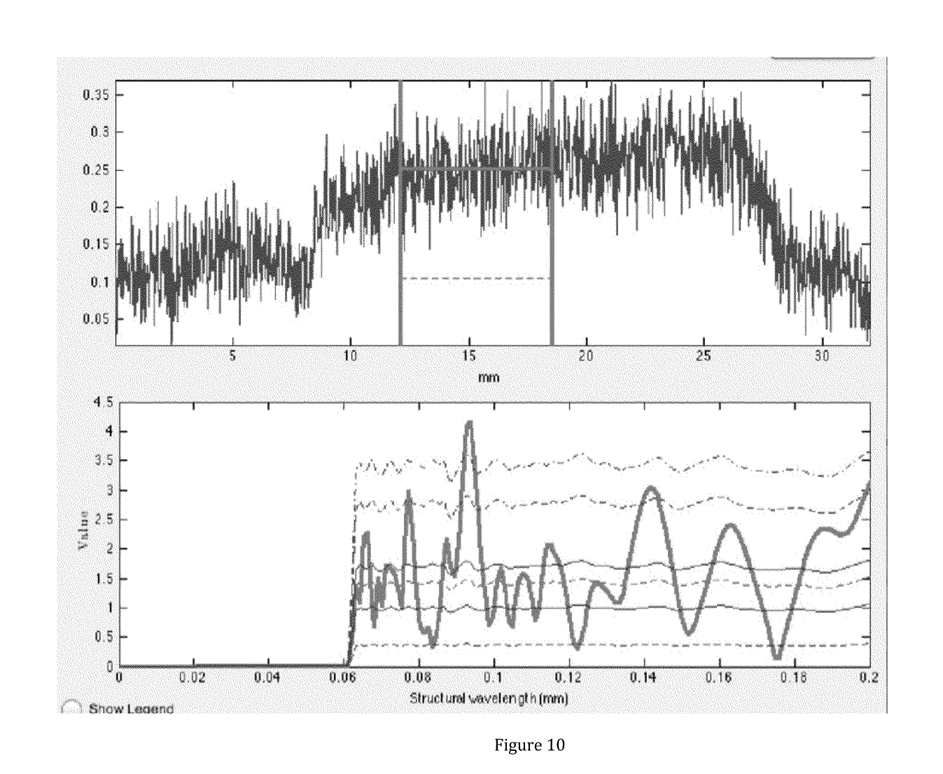

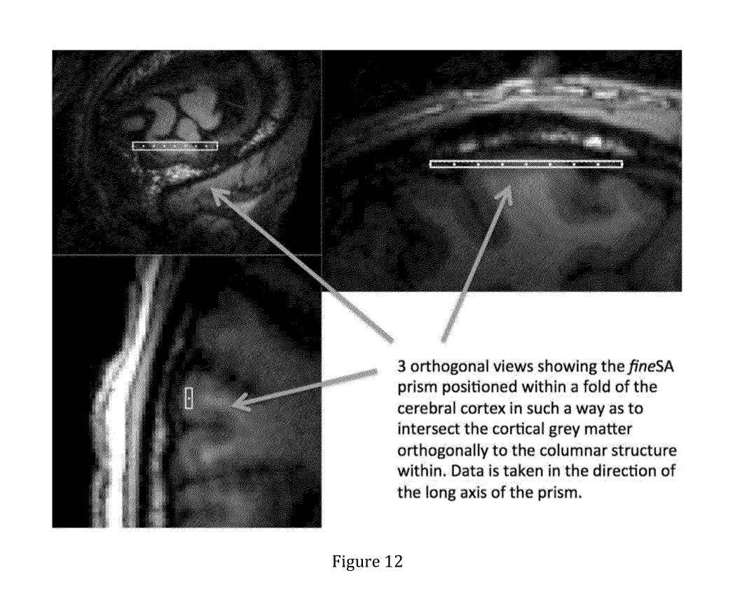

An example of a possible prism placement within brain tissue for applying the prior art magnetic resonance fine texture measurement technique to measurement of the spacing and regularity of cortical structures such as minicolumns is shown in FIG. 12. Similar prism positioning can be used in application of this technique to assessment and monitoring of cortical structures seen in healthy and in cognitively impaired subjects as a measure of health/pathology. FIG. 5 depicts structural spectra generated from histology images of cortical structure as a depiction of one target of the technique.

Although the prior art magnetic resonance fine texture measurement technique is relatively insensitive to motion as long as the acquisition volume remains in a relatively homogenous region of tissue, measurement in regions of very small extent can be problematic due to motion out of the region. Certain of the refinements to the prior art magnetic resonance fine texture measurement technique discussed above relate to assuring that the acquisition volume remains within the tissue of interest during data acquisition. An envisaged refinement to the technique would be to actively track patient motion, using for example, accelerometers, interferometers, cameras, or other sensing equipment and software, and actively adjust the positioning of the acquisition volume using this measured information.

Another use of the-above described adaptations of the magnetic resonance fine texture measurement technique to use in brain pathology is to map out the boundaries of the various control regions of the cortex, in vivo, as part of functional or other brain studies (brain conditions). This can be accomplished by acquiring data at adjacent regions of the cortex in the general vicinity where these boundaries are expected to lie, and look for spatial changes in the resultant spectra indicative of a structural change occurring at the boundary. Use of small ROIs in data analysis would enable high precision localization of control region boundaries. Two possible methods envisaged would be to monitor these changes with the readout direction positioned 1) parallel to the cortex or 2) perpendicular to the cortex, looking for changes in spectral signature indicative of a boundary.

These adaptations of the prior art magnetic resonance fine texture measurement technique to facilitate its use in brain pathology can be used singly or in combination to assess/diagnose and monitor tissue changes in any region in the brain in response to a range of diseases and pathologies, changes associated with traumatic brain injury, as well as in studies of brain function.

The refinements in the basic MR-based technique facilitate monitoring changes over time as pathology progresses and symptoms intensify, or as therapies provide amelioration of symptoms.

Thus the present invention has a number of aspects, which aspects may be practiced alone or in various combinations or sub-combinations, as desired. While a preferred embodiment of the present invention has been disclosed and described herein for purposes of illustration and not for purposes of limitation, it will be understood by those skilled in the art that various changes in form and detail may be made therein without departing from the spirit and scope of the invention as defined by the full breadth of the following claims.

What is claimed is:

1. A method of assessing conditions or disease of a brain in a patient comprising:
acquiring spatially-encoded MR echoes along a acquisition axis of a selectively-excited internal volume positioned within a targeted region in a patient's brain while applying a magnetic field gradient;
analyzing the spatially-encoded MR echoes along an acquisition axis in the selectively-excited internal volume to yield a spectrum of textural wavelengths in a region of interest along a spatially-encoded axis of the internal volume;
characterizing and assessing the conditions or disease from the region of interest and the spectrum of textural wavelengths in the region of interest in comparison to known spectrums of textural wavelengths in a corresponding region of interest taken from the same or different patients.

2. The method of claim 1 wherein the selectively-excited internal volume is positioned within the patient's cortex.

3. The method of claim 1 wherein the method is repeated at multiple times and for multiple regions of interest in the patient's cortex to assess the spatial and temporal progression of the brain disease.

4. The method of claim 1 wherein the selectively-excited internal volume is positioned to run along a top of a cortical fold.

5. The method of claim 1 wherein the selectively-excited internal volume is positioned to run along a side of a cortical fold.

6. The method of claim 1 wherein the selectively-excited internal volume is positioned to run along a bottom of a cortical fold.

7. The method of claim 1 further comprising:
positioning the selectively-excited internal volume along a curve in the patient's cortex in a region of interest containing non-isotropic repeating structures and with the acquisition axis oriented to intersect the structures at angles either side of orthogonal so that different regions of interest along the acquisition axis in the selectively-excited internal volume have different angles with respect to the magnetic field gradient; and
comparing the textural wavelengths from different regions of interest along the selectively-excited internal volume;
thereby providing verification of which part of a structural spectrum arises from columnar organization.

8. The method of claim 1 for assessment of ordered, non-isotropic textures further comprising:
acquiring successive spatially-encoded MR echoes at a range of readout gradient angles relative to the acquisition axis of the selectively-excited internal volume.

9. The method of claim 1 wherein acquiring spatially-encoded MR echoes along the spatially-encoded axis of a selectively-excited internal volume comprises using partial Fourier acquisition of a symmetric spin echo to allow a shorter echo time and hence a stronger signal at k values of interest;
thereby acquiring contrast data in regions of the brain between high fat content materials and high water content materials.

10. The method of claim 9 wherein artifacts present on a leading portion of the spatially-encoded MR echoes are avoided by using a trailing portion of the echo.

11. The method of claim 1 to highlight vasculature wherein:
acquiring spatially-encoded MR echoes along the spatially-encoded axis of a selectively-excited internal volume comprises using a spin echo wherein k values of interest fall at a time displaced from spin echo time;
whereby contrast develops between blood in the vasculature and surrounding tissue.

12. The method of claim 11 wherein artifacts present on a leading portion of the spatially-encoded MR echoes are avoided by using a trailing portion of the echo.

13. The method of claim 11 wherein the k values of interest on the trailing part of the echo are produced at an earlier time to allow better signal to noise for highlighting contrast between myelinated axons and surrounding tissue;
thereby providing a higher signal by use of a partial Fourier echo.

14. The method of claim 11 wherein putting the spin echo before k0 allows greater development of contrast between vasculature and surrounding tissue by the time high-frequency k values of interest are recorded, and before T2 decay has significantly reduced the echo signal.

15. The method of claim 11 wherein the spin echo is positioned as close as possible to the k values of interest to highlight contrast between inflammatory structure and surrounding tissue.

16. The method of claim 1 further comprising, when acquiring the spatially-encoded MR echoes and the selectively-excited internal volume is near the patient's skull, using a surface coil in proximity to the patient's head to acquire the spatially-encoded MR echoes.

17. The method of claim 1 further comprising:
positioning the selectively-excited internal volume with its acquisition axis traversing a curved section of the patient's cortex to ensure that the acquisition axis aligns with minicolumn structures of the patient's cortex at different angles along the acquisition axis through the cortex; and
using the observed variation in spectrum with angle to calculate the columnar spacing and width and to obtain information on a degree of order of the minicolumn structure as an additional measure of disease advancement.

18. The method of claim 1 wherein the magnetic field gradient direction intersects repeating structures at 90° and at angles on either side of 90°.

19. The method of claim 18 wherein the repeating structures are cortical minicolumns.

20. The method of claim 19 wherein assessing a disease of the brain includes assessing changes in the organization of cortical minicolumns.

21. The method of claim 19 wherein assessing a disease of the brain includes assessing changes in the organization of cortical minicolumns as part of assessing autism and schizophrenia.

22. The method of claim 19 wherein assessing a disease of the brain includes assessing changes in the organization of cortical minicolumns in AD onset and progression.

23. The method of claim 1 wherein the method is used to diagnose and assess dementia-causing brain disease.

24. The method of claim 1 wherein characterizing the disease includes distinguishing between dementia-causing brain diseases.

25. The method of claim 1 wherein assessing a disease of the brain includes assessing a progression of the disease.

26. The method of claim 1 wherein assessing a disease of the brain includes assessing temporal and spatial progression of the disease.

27. The method of claim 1 wherein assessing a disease of the brain includes assessing amyloid beta plaque deposition and attendant tissue changes in brain tissue and within vasculature.

28. The method of claim 1 wherein assessing a disease of the brain includes assessing changes in microvasculature within the patient's cortex and underlying white matter in response to onset of dementia.

29. The method of claim 1 wherein assessing a disease of the brain includes assessing of changes in white matter.

30. The method of claim 1 wherein assessing a disease of the brain includes assessing changes in white matter attendant with Multiple Sclerosis.

31. The method of claim 1 wherein assessing a disease of the brain includes assessing changes in vasculature and surrounding tissue in response to development of cerebrovascular disease.

32. The method of claim 1 wherein assessing a disease of the brain includes assessing inflammatory effects in tissue attendant with disease development.

33. The method of claim 1 wherein assessing a disease of the brain includes assessing tissue textural/structural changes attendant in development and progression of brain disease.

34. The method of claim 1 wherein assessing conditions of the brain includes determining boundaries of control regions in the patient's cortex in vivo for use in measurements of brain function in both diseased and healthy brains.

35. The method of claim 1 further comprising using a head cradle for patient stabilization.

36. The method of claim 1 further comprising using a surface coil for high gain.

37. The method of claim 1 further comprising using real-time measurement of and correction for patient motion.

38. The method of claim 1 further comprising using repeated acquisition of 3D reference images while acquiring spatially-encoded MR echoes along the spatially-encoded axis of a selectively-excited internal volume positioned within a targeted region in a patient's brain while applying a magnetic field gradient in order to monitor and correct for patient motion.

39. The method of claim 1 further comprising tailoring a cross-section of the selectively-excited internal volume to fit within a selected tissue region.

40. The method of claim 1 further comprising using PASE, PEASE, or PSSE acquisition sequences.

41. The method of claim 1 further comprising using exogenous or endogenous contrast.

42. The method of claim 1 further comprising monitoring of the spatial and temporal progression of disease effects in the brain to identify the disease and determine its progression.

43. The method of claim 1 further comprising monitoring tissue changes in both gray matter and white matter attendant with brain atrophy development and variation in tissue MR signal intensity due to aging and disease.

44. The method of claim 1 further comprising applying varying angles of the magnetic field gradient for acquisition of successive echoes during a measurement series.

45. The method of claim 1 further comprising:
positioning the axis of the selectively-excited internal volume along a region of interest where an organized structure curves; and,
measuring variations in structural spectrum along a curve of the organized structure.
